(19) United States
(12) Patent Application Publication
(10) Pub. No.: US 2025/0115849 A1
(43) Pub. Date: Apr. 10, 2025

(54) CULTURE SYSTEM

(71) Applicants: Canon Kabushiki Kaisha, Tokyo (JP); CANON MEDICAL SYSTEMS CORPORATION, Tochigi (JP)

(72) Inventors: Tomoki HANEISHI, Kita-ku (JP); Tetsuya SAKAI, Ryugasaki (JP)

(73) Assignees: Canon Kabushiki Kaisha, Tokyo (JP); CANON MEDICAL SYSTEMS CORPORATION, Tochigi (JP)

(21) Appl. No.: 18/907,298

(22) Filed: Oct. 4, 2024

(30) Foreign Application Priority Data
Oct. 6, 2023 (JP) 2023-174553

(51) Int. Cl. C12M 1/00; C12M 1/107; C12M 1/12; C12M 3/06

(52) U.S. Cl. CPC C12M 29/00; C12M 23/06; C12M 23/36; C12M 27/16

(57) ABSTRACT

A culture system according to the present embodiment includes a supply unit, a discharge unit, a collection unit, and a housing. The supply unit supplies a culture vessel with a culture medium for culturing of cells. The discharge unit discharges liquid droplets for detaching the cells placed in the culture vessel. The collection unit that collects the cells which have been detached in the culture vessel. The housing has the culture vessel, the supply unit, the discharge unit, and the collection unit disposed therein. In the state in which the supply unit has been moved away from the fluid level of the culture medium, the housing is tilted to position the collection unit at a lower level than the supply unit, and then the culture medium and the cells are collected using the collection unit.

### CROSS-REFERENCE TO RELATED APPLICATIONS

This application is based upon and claims the benefit of priority from Japanese Patent Application No. 2023-174553, filed on Oct. 6, 2023; the entire contents of which are incorporated herein by reference.

### FIELD

Embodiments described herein relate generally to a culture system.

### BACKGROUND

There are two types of cell culturing, namely, open-type culturing and closed-type culturing. The open-type culturing is a commonly-used culturing method in which, usually, culturing is performed using a dish representing a culture vessel, and tasks such as culture medium replacement and passage culture are performed by the worker by opening the lid of the dish. In the open-type culturing, the space used for cell culturing is basically in the open condition. Hence, the worker is highly likely to get exposed to bacterial contamination; and, in the case of using a virus vector, there is a high infection risk to the worker. Thus, there are issues in regard to the safety. In the closed-type culturing, although cell culturing can be performed in a closed-type (sealed) culture vessel, the tasks such as culture medium replacement and cell detachment are performed by the worker in the open condition. Hence, there is a risk of infection. For that reason, there is a demand for a culture apparatus that enables performing culture medium replacement and cell detachment too in the closed condition. Moreover, also regarding the task of collecting the cells that have been detached inside the culture vessel, there is a demand for being able to perform that task in the closed condition.

### BRIEF DESCRIPTION OF THE DRAWINGS

FIG. 1 is a perspective view illustrating a configuration of a culture apparatus included in a culture system according to an embodiment;

FIGS. 2 and 3 are sectional side views illustrating a configuration of the culture apparatus included in the culture system according to the present embodiment;

FIG. 4A is a sectional side view illustrating a configuration of the culture apparatus included in the culture system according to the present embodiment, and is a diagram illustrating the state in which a supply unit (a tube for cell seeding and medium replacement) has been moved away from the fluid level of the culture medium;

FIG. 4B is a sectional side view illustrating a configuration of the culture apparatus included in the culture system according to the present embodiment, and is a diagram illustrating that, in the state in which the supply unit (a tube for cell seeding and medium replacement) has been moved away from the fluid level of the culture medium, a housing is tilted to position a collection unit (a tube for waste solution collection and a tube for cell suspension liquid collection) at a lower level than the supply unit;

FIGS. 5A and 5B are sectional side views illustrating a configuration of the culture system according to the present embodiment, and are diagrams for explaining a tilting mechanism;

FIGS. 6A and 6B are sectional side views illustrating a configuration of the culture system according to the present embodiment, and are diagrams for explaining a cartridge in which culture apparatuses are housed;

FIG. 7A is a sectional side view illustrating a configuration of a culture system according to the present embodiment, and is a diagram for explaining an incubator in which the cartridge is housed; and

FIG. 7B is a perspective view illustrating a configuration of a culture system according to the present embodiment, and is a diagram for explaining the incubator in which the cartridge is housed.

### DETAILED DESCRIPTION

A culture system according to an embodiment includes a supply unit, a discharge unit, a collection unit, and a housing. The supply unit supplies a culture vessel with a culture medium for culturing of cells. The discharge unit discharges liquid droplets for detaching the cells placed in the culture vessel. The collection unit that collects the cells which have been detached in the culture vessel. The housing has the culture vessel, the supply unit, the discharge unit, and the collection unit disposed therein. In the state in which the supply unit has been moved away from the fluid level of the culture medium, the housing is tilted to position the collection unit at a lower level than the supply unit, and then the culture medium and the cells are collected using the collection unit.

An exemplary embodiment of a culture apparatus is described below in detail with reference to the accompanying drawings. However, the embodiments are not limited to the embodiment described below. Moreover, the details given in a single embodiment are applicable in principle to other embodiments too in an identical manner.

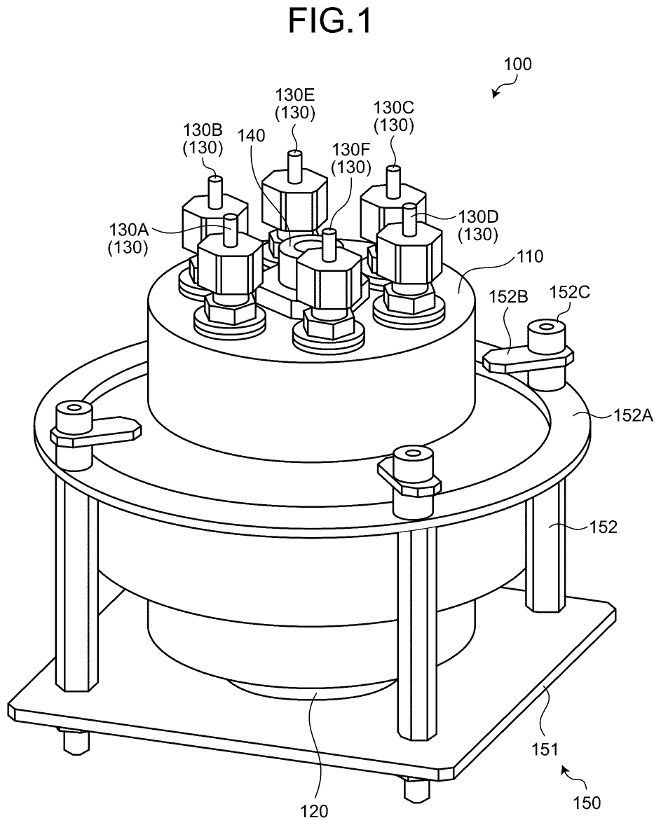

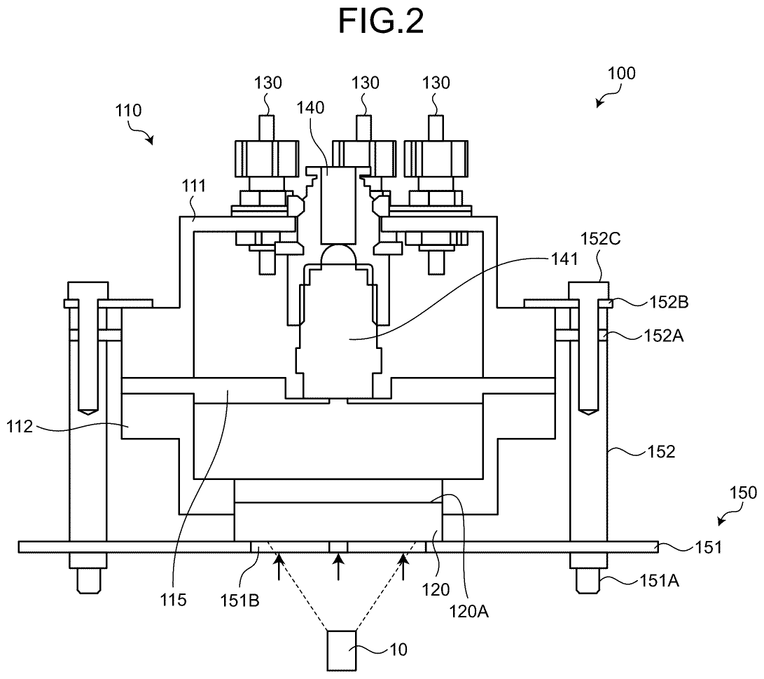

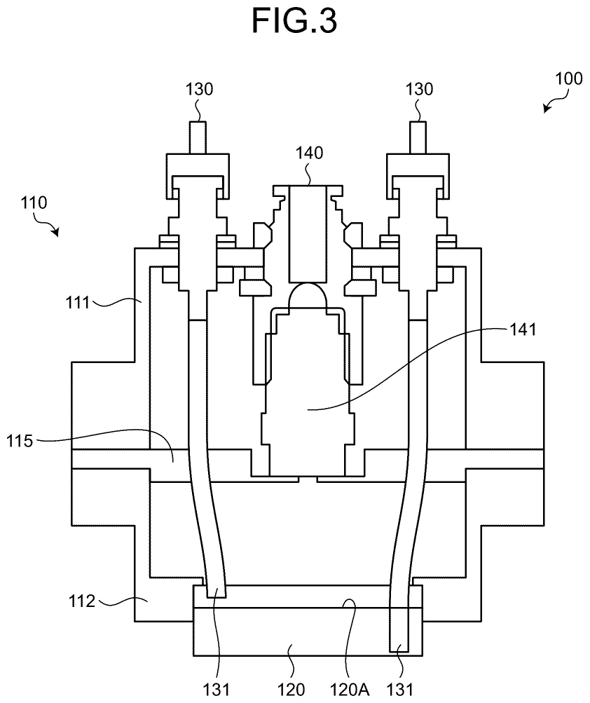

FIG. 1 is a perspective view illustrating a configuration of a culture apparatus 100 included in a culture system according to the present embodiment. FIGS. 2 and 3 are sectional side views illustrating a configuration of the culture apparatus 100 included in the culture system according to the present embodiment. In FIG. 2, tubes 131 illustrated in FIG. 3 are omitted. In FIG. 3, a support member 150 illustrated in FIGS. 1 and 2 is omitted.

As illustrated in FIG. 1, the culture system according to the present embodiment includes the culture apparatus 100. The culture apparatus 100 includes a housing 110, a light shielding plate 115, and a plurality of couplings 130.

The housing 110 has a cylindrical portion in which tubes (explained later) are disposed; and includes a first housing portion 111 and a second housing portion 112 that is disposed in the lower part of the first housing portion 111. The light shielding plate 115 is disposed in between the first housing portion 111 and the second housing portion 112. In the culture apparatus 100 according to the present embodiment, an arbitrary sealing mechanism is disposed in between the first housing portion 111 and the light shielding plate 115 as well as in between the light shielding plate 115 and the second housing portion 112. As a result, the sealing property of the housing 110 is secured.

The first housing portion 111, the light shielding plate 115, and the second housing portion 112 have, for example, screw holes formed thereon in the vertical direction and are screw-clamped together. That is, the first housing portion 111 and the second housing portion 112 support the light shielding plate 115.

In the culture apparatus 100, in order to reduce the infection risk to the worker, tubes are used for enabling culture medium replacement and cell collection in the closed condition.

In the upper part of the first housing portion 111, the plurality of couplings 130 are disposed. As illustrated in FIG. 3, the culture apparatus 100 further includes a plurality of tubes 131. Each of the plurality of tubes 131 is disposed inside the housing 110 via one of the plurality of couplings 130.

For example, the plurality of tubes 131 include a tube for cell seeding and culture medium replacement, a tube for waste solution collection, a tube for PBS addition, a tube for EDTA addition, a tube for cell suspension liquid collection, and a spare tube. In the example illustrated in FIG. 1, the plurality of couplings 130 include a coupling 130A for the tube 131 for cell seeding and culture medium replacement; a coupling 130B for the tube 131 for waste solution collection; a coupling 130C for the tube 131 for PBS addition; a coupling 130D for the tube 131 for EDTA addition; a coupling 130E for the tube 131 for cell suspension liquid collection; and a coupling 130F for the spare tube 131.

As illustrated in FIG. 3, inside the housing 110, a plurality of tubes 131 is disposed. As illustrated in FIG. 3, on the light shielding plate 115, openings are formed through which the plurality of tubes 131 are disposed.

As illustrated in FIGS. 2 and 3, in the bottom part of the second housing portion 112, a culture vessel 120 is disposed that represents a dish for culturing cells. The culture vessel 120 has a circular shape. On top of the culture vessel 120, the culture apparatus 100 is placed as a lid. Inside the culture vessel 120, the leading ends of the plurality of tubes 131 are disposed.

In the culture apparatus 100, a nozzle is used for performing cell detachment in the closed condition.

As illustrated in FIGS. 1 to 3, the culture apparatus 100 further includes a coupling 140. Moreover, as illustrated in FIGS. 2 and 3, the culture apparatus 100 further includes a detachment nozzle 141.

The coupling 140 is disposed in the central portion of the upper part of the first housing portion 111. The detachment nozzle 141 is disposed inside the housing 110 and is connected to the coupling 140 in the upper part of the first housing portion 111. In the central portion of the light shielding plate 115, an opening is formed into which the leading end of the detachment nozzle 141 is disposed.

As illustrated in FIG. 1, the culture apparatus 100 and the culture vessel 120 are supported by the support member 150.

The support member 150 includes a first-type support member 151 and second-type support members 152. The first-type support member 151 supports the culture vessel 120 that is disposed in the bottom part of the second housing portion 112; and the culture vessel 120 is disposed in the central portion of the first-type support member 151.

For example, the second-type support members 152 are columnar in shape, and the first-type support member 151 has through holes formed thereon for allowing passage of the second-type support member 152. Moreover, in each second-type support member 152, a member 152A for supporting the corresponding side face of the housing 110 is disposed, and the outer diameter of the member 152A corresponds to the inner diameter of the housing 110. At the upper end of each second-type support member 152, a member 152B is disposed for pressing the first housing portion 111 from the upper side. Herein, screws 152C are inserted from the upper end of the second-type support members 152, nuts 151A are put in from the bottom face of the first-type support member 151 on which the through holes are formed, and the first-type support member 151 is screw-clamped to the second-type support members 152. That provides support to the culture apparatus 100 and the culture vessel 120.

The following explanation is given about the role of the light shielding plate 115.

For example, the worker observes the inside of the culture vessel 120 using a camera. As illustrated in FIG. 2, in the central portion of the first-type support member 151, an observation window 151B is provided for enabling the worker to observe the inside of the culture vessel 120 using a camera 10. The light shielding plate 115 that is disposed inside the housing 110 is provided for enabling observation using the camera 10; and, when the culture vessel 120 is illuminated from the side, is used to prevent the detachment nozzle 141 and the tubes 131 from being photographed.

The following explanation is given about the role of the plurality of tubes 131 and the detachment nozzle 141.

In the culture apparatus 100, the detachment nozzle 141 is used because no lid is provided for enabling culture medium replacement and cell detachment in the closed condition. In the culture apparatus 100, for example, phosphate-buffered saline (PBS) is sprayed as the liquid droplets from the detachment nozzle 141; and the cells adhered to the bottom of the culture vessel 120 are detached, and the detached cells are sucked in and collected through the tube 131 for cell suspension liquid collection.

More particularly, in the culture apparatus 100, firstly, a process is performed in which cells are seeded in the culture vessel 120 using the tube 131 for cell seeding and culture medium replacement. Then, in the culture apparatus 100, a process is performed in which the culture medium in the culture vessel 120 is replaced using the tube 131 for cell seeding and culture medium replacement and the tube 131 for waste solution collection. Since the cells inside the culture vessel 120 are unclean due to the waste matter such as the culture medium, in the culture apparatus 100, a process is performed in which the cells inside the culture vessel 120 are cleaned by adding PBS as a cleaning fluid using the tube 131 for PBS addition, and then the PBS is collected using the tube 131 for waste solution collection. Subsequently, in the culture apparatus 100, a process is performed in which the adhesion force among the cells and the adhesion force between the cells and the culture vessel 120 is weakened by adding ethylenediaminetetraacetic acid (EDTA) as a liquid solution into the culture vessel 120 using the tube 131 for EDTA addition, and then the EDTA is collected using the tube 131 for waste solution collection. Subsequently, in the culture apparatus 100, a process is performed in which PBS liquid droplets are sprayed into the culture vessel 120 using the detachment nozzle 141, so that the water droplets are applied inside the culture vessel 120 and the cells adhered to the bottom of the culture vessel 120 are detached. Lastly, in the culture apparatus 100, a process is performed in which the cells that have been detached inside the culture vessel 120 are sucked in and collected using the tube 131 for cell suspension liquid collection. At that time, a culture medium is added using the tube 131 for medium replacement, and the added culture medium and the cells are collected using the tube 131 for waste solution collection and the tube 131 for cell suspension liquid collection. All the abovementioned processes are performed in the closed condition.

Meanwhile, the tube 131 for cell seeding and culture medium replacement represents an example of a “supply unit”. The detachment nozzle 141 represents an example of a “discharge unit”. The tube 131 for waste solution collection and the tube 131 for cell suspension liquid collection represent an example of a “collection unit”.

In the culture apparatus 100, in order to perform culture medium replacement and cell detachment in the closed condition, the leading ends of the plurality of tubes 131 are disposed inside the culture vessel 120. That is, the leading ends of the plurality of tubes 131 are immersed below the fluid level inside the culture vessel 120. Hence, when the cells that have been detached inside the culture vessel 120 are sucked in and collected using the tube 131 for cell suspension liquid collection; for example, the detached cells get attached to the leading ends of the tubes 131 such as the tube 131 for cell seeding and culture medium replacement. That results in a loss in the collection of cells. In that regard, there is a demand for a culture system having high cell collection efficiency.

In that regard, in order to enhance the cell collection efficiency during cell culturing in the closed condition, the culture system according to the present embodiment includes the supply unit (the tube 131 for cell seeding and medium replacement), the discharge unit (the detachment nozzle 141), the collection unit (the tube 131 for waste solution collection and the tube 131 for cell suspension liquid collection), and the housing 110. The supply unit supplies the culture vessel 120 with a culture medium for cell culturing. The discharge unit discharges the liquid droplets for detaching the cells placed in the culture vessel 120. The collection unit (in this case, the tube 131 for cell suspension liquid collection) collects the cells that have been detached inside the culture vessel 120. The housing 110 has the culture vessel 120, the supply unit, the discharge unit, and the collection unit disposed therein. In the culture system according to the present embodiment, in the state in which the supply unit has been moved away from the fluid level of the culture medium, the housing 110 is tilted in such a way that the collection unit is positioned at a lower level than the supply unit, and then the culture medium and the cells are collected using the collection unit.

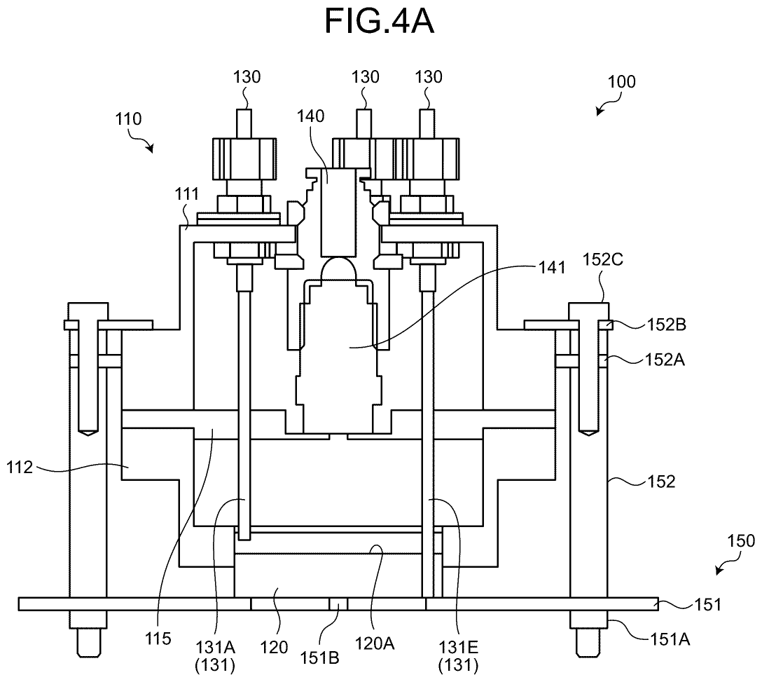

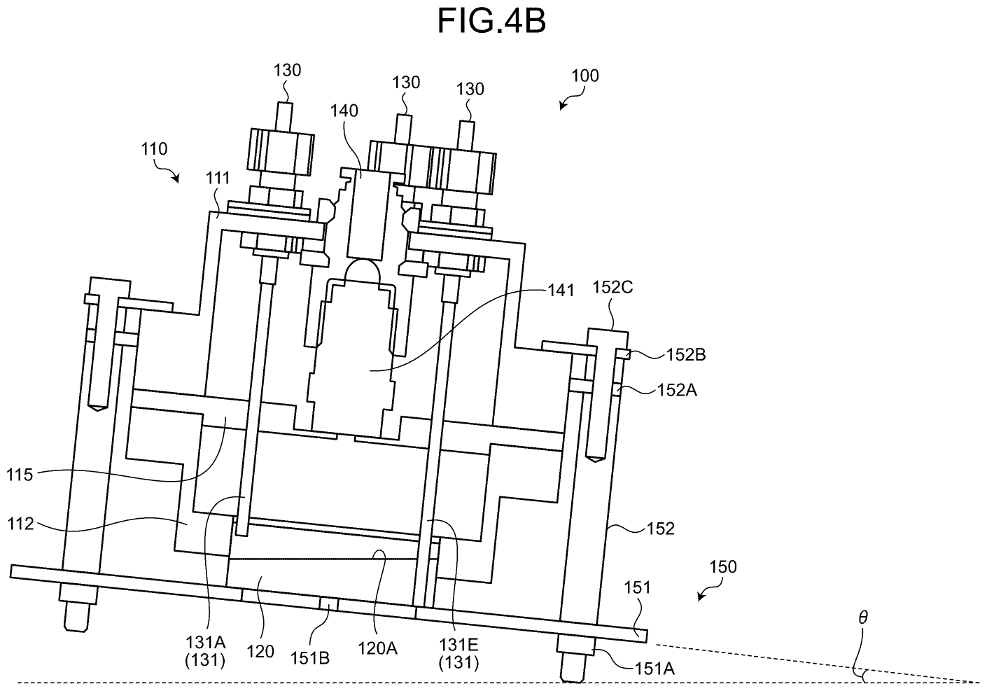

FIGS. 4A and 4B are sectional side views illustrating a configuration of the culture apparatus 100 included in the culture system according to the present embodiment. In the housing 110, the supply unit (the tube 131 for cell seeding and medium replacement) and the collection unit (the tube 131 for waste solution collection and the tube 131 for cell suspension liquid collection) are disposed in the directions facing each other.

Herein, the tube 131 for cell seeding and culture medium replacement, the tube 131 for waste solution collection, the tube 131 for PBS addition, the tube 131 for EDTA addition, the tube 131 for cell suspension liquid collection, and the spare tube 131 are disposed inside the housing 110 via the couplings 130A to 130F, respectively. In the example illustrated in FIGS. 4A and 4B, the tube 131 for cell seeding and culture medium replacement is illustrated as a “tube 131A for cell seeding and culture medium replacement”, and the tube 131 for cell suspension liquid collection is illustrated as a “tube 131E for cell suspension liquid collection”. FIG. 4A is a diagram illustrating the state in which the supply unit (the tube 131 for cell seeding and medium replacement) has been moved away from a fluid level 120A of the culture medium. In the examples illustrated in FIGS. 3 and 4A, the discharge outlet of the supply unit (the leading end of the tube 131 for cell seeding and medium replacement) has been moved away from the fluid level 120A of the culture medium.

FIG. 4B is a diagram in which, in the state in which the supply unit (the leading end of the tube 131 for cell seeding and medium replacement) has been moved away from the fluid level 120A of the culture medium, the housing 110 is tilted in such a way that the collection unit (the tube 131 for waste solution collection and the tube 131 for cell suspension liquid collection) is positioned at a lower level than the supply unit. For example, a tilt angle θ of the housing 110 with respect to the horizontal plane is equal to or greater than 1° and equal to or smaller than 60°. In the culture system according to the present embodiment, in the state in which the discharge outlet of the supply unit (the leading end of the tube 131 for cell seeding and medium replacement) has been moved away from the fluid level 120A of the culture medium, the housing 110 is tilted in such a way that the inlet of the collection unit (the leading end of the tube 131 for waste solution collection and the leading end of the tube 131 for cell suspension liquid collection) is positioned at a lower level than the discharge outlet of the supply unit. At that time, the cell suspension liquid moves toward the collection unit. Then, in the culture system according to the present embodiment, in the state in which the housing 110 is tilted by the tilt angle θ with respect to the horizontal plane, the culture medium and the cells are collected using the collection unit. As a result, in the culture system according to the present embodiment, when performing cell culturing in the closed condition, the culture medium and the cells are collected only after the cell suspension liquid has been moved toward the collection unit. That enables achieving enhancement in the cell collection efficiency.

In the example illustrated in FIGS. 4A and 4B, even before tilting the housing 110, the discharge outlet of the supply unit has been moved away from the fluid level 120A of the culture medium. However, that is not the only possible case. Alternatively, for example, after tilting the housing 110, the discharge outlet of the supply unit can be moved away from the fluid level 120A of the culture medium.

In the culture system according to the present embodiment, in order to further enhance the cell collection efficiency, the culture medium can be again added and the added culture medium can be collected. For example, after the culture medium and the cells are collected using the collection unit (the tube 131 for waste solution collection and the tube 131 for cell suspension liquid collection), the culture medium is added to the culture vessel 120 by the supply unit (the tube 131 for cell seeding and medium replacement). Then, the added culture medium is further collected by the collection unit (in this case, the tube 131 for waste solution collection).

The following explanation is given about a mechanism for tilting the culture apparatus 100.

The culture system according to the present embodiment further includes a tilting mechanism. In the state in which the supply unit (the leading end of the tube 131 for cell seeding and medium replacement) has been moved away from the fluid level 120A of the culture medium, the tilting mechanism tilts the culture apparatus 100 in such a way that the collection unit (the tube 131 for waste solution collection and the tube 131 for cell suspension liquid collection) is positioned at a lower level than the supply unit.

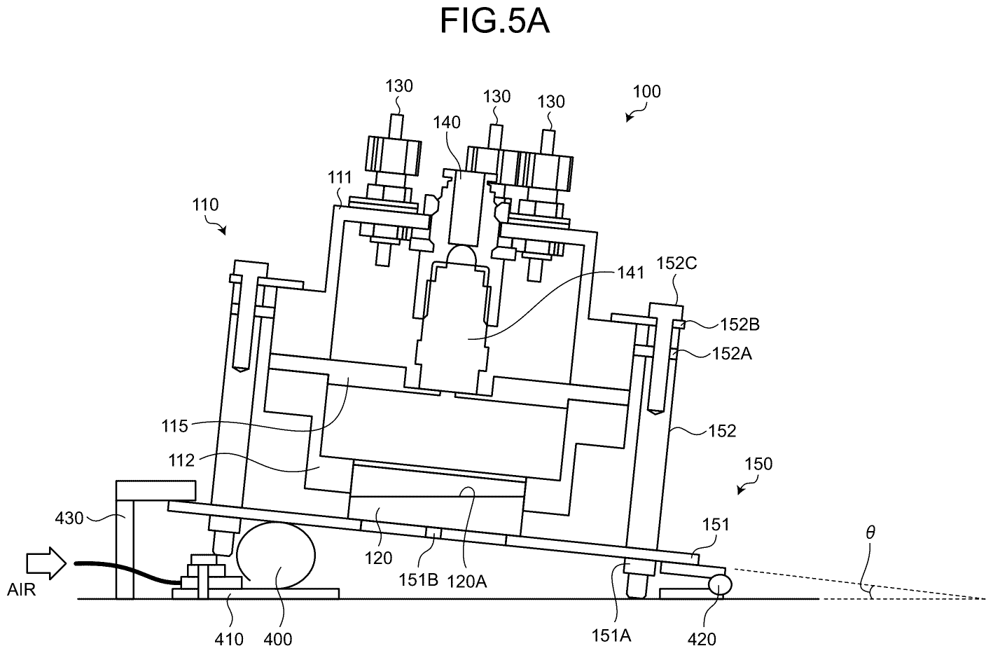

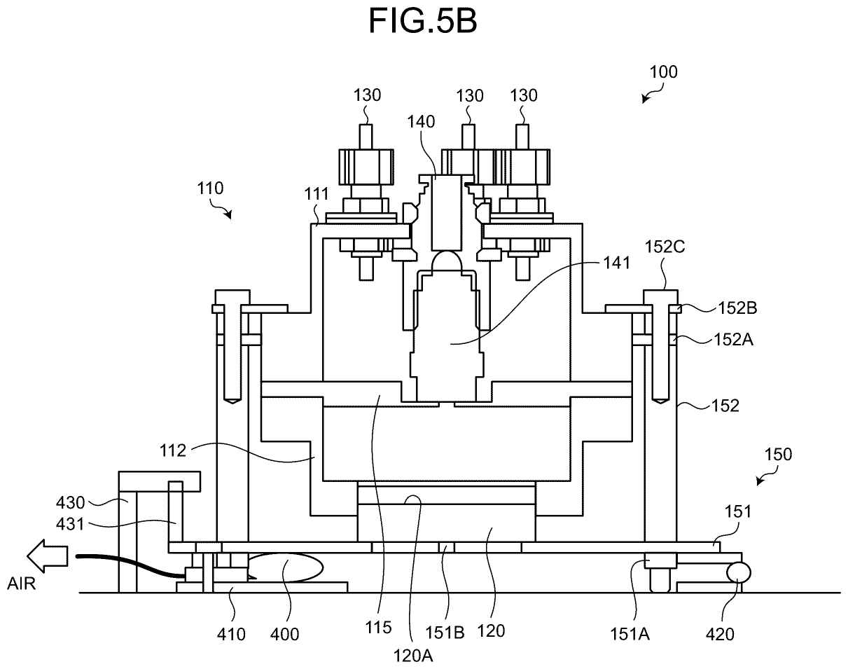

FIGS. 5A and 5B are sectional side views illustrating a configuration of the culture system according to the present embodiment, and are diagrams for explaining the tilting mechanism. In FIGS. 5A and 5B, the tubes 131 illustrated in FIG. 4 are omitted.

FIG. 5A is a diagram illustrating the operation by which the tilting mechanism tilts the culture apparatus 100. In the example illustrated in FIG. 5A, the tilting mechanism includes a balloon member 400, support members 410 and 420, and a stopper member 430. The balloon member 400 is disposed on the bottom face of the culture apparatus 100. The support member 410 includes a path for admitting and releasing air, and supports the balloon member 400. Thus, when the air flows into the balloon member 400 via the support member 410, the balloon member 400 disposed on the bottom face of the culture apparatus 100 becomes inflated, and the inflated balloon causes tilting of the culture apparatus 100 by the angle θ with respect to the horizontal plane. The support member 420 is a hinged member, and supports the tilted culture apparatus 100 with the hinge serving as the pivot point. The stopper member 430 is provided to stop the tilting of the culture apparatus 100.

In this way, in the culture system according to the present embodiment, when the air is supplied to the balloon member 400 disposed on the bottom face of the culture apparatus 100, the tilting mechanism tilts the culture apparatus 100.

FIG. 5B is a diagram illustrating the state in which the operation performed by the tilting mechanism is cancelled. In the example illustrated in FIG. 5B, the tilting mechanism further includes a coil spring 431. The coil spring 431 is connected to the stopper member 430 and the first-type support member 151. When the air inside the balloon member 400 is taken out, the amount of lift is reduced and the culture apparatus 100 is supported at the horizontal level with the strength of the coil spring 431. Alternatively, at the time of taking the air out from the balloon member 400 via the support member 410, the coil spring 431 can press the first-type support member 151 so that the balloon member 400 is deflated and the culture apparatus 100 is supported at the horizontal level.

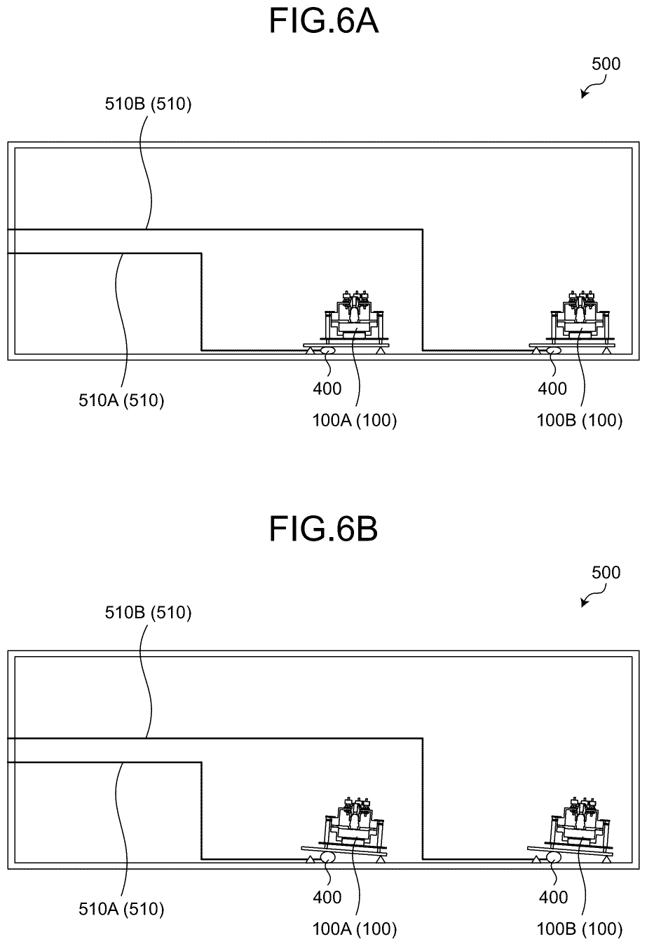

FIGS. 6A and 6B are sectional side views illustrating a configuration of the culture system according to the present embodiment, and are diagrams for explaining a cartridge 500 in which the culture apparatuses 100 are housed.

As illustrated in FIG. 6A, the culture system according to the present embodiment further includes the cartridge 500 in which the culture apparatuses 100 are housed. The cartridge 500 includes: a flow path for supplying the culture medium to the supply unit (the tube 131 for cell seeding and medium replacement); a flow path for supplying liquid droplets to the discharge unit (the detachment nozzle 141); a flow path for collecting the cells from the collection unit (the tube 131 for waste solution collection and the tube 131 for cell suspension liquid collection); and a flow path 510 for supplying air to the balloon member 400.

FIG. 6A is a diagram illustrating the state in which a culture apparatus 100A for expansive culturing and a culture apparatus 100B for main culturing are housed in the cartridge 500. Thus, in the cartridge 500, the culture apparatus 100A for expansive culturing and the culture apparatus 100B for main culturing are housed as the culture apparatuses 100.

For example, culturing is carried out twice and, inside the cartridge, the stage of culturing is different in the expansive culturing than in the main culturing. In the example of culturing iPS cells; as expansive culturing, cells that are in a prior state to becoming iPS cells are cultured; and, after expansive culturing is performed, iPS cells are formed by performing gene transfer. As main culturing, culturing is performed to increase the cells that have turned into iPS cells.

In that case, regarding the culture apparatus 100A for expansive culturing, the cartridge 500 includes: a flow path for supplying the culture medium to the supply unit; a flow path for supplying liquid droplets to the discharge unit; a flow path for collecting the cells using the collection unit; and a flow path 510A for supplying air to the balloon member 400. For example, the air is supplied using, for example, a pump disposed outside the cartridge 500. In an identical manner, for the culture apparatus 100B for main culturing, the cartridge 500 includes: a flow path for supplying the culture medium to the supply unit; a flow path for supplying liquid droplets to the discharge unit; a flow path for collecting the cells using the collection unit; and a flow path 510B for supplying air to the balloon member 400. With reference to FIG. 6A, the balloon member 400 disposed on the bottom face of each of the culture apparatuses 100A and 100B is in the deflated state. Hence, the culture apparatuses 100A and 100B are maintaining the horizontal level in the cartridge 500.

FIG. 6B is a diagram illustrating the state in which the culture apparatus 100A for expansive culturing and the culture apparatus 100B for main culturing are tilted inside the cartridge 500. When the air flows into the balloon members 400 via the flow paths 510A and 510B, the balloon members 400 disposed on the bottom faces of the culture apparatuses 100A and 100B become inflated; and the inflated balloon members 400 cause tilting of the culture apparatuses 100A and 100B. That is, the culture apparatuses 100A and 100B become tilted inside the cartridge 500. Thus, in the culture system according to the present embodiment, the culture medium and the cells are collected in the tilted state of the culture apparatuses 100.

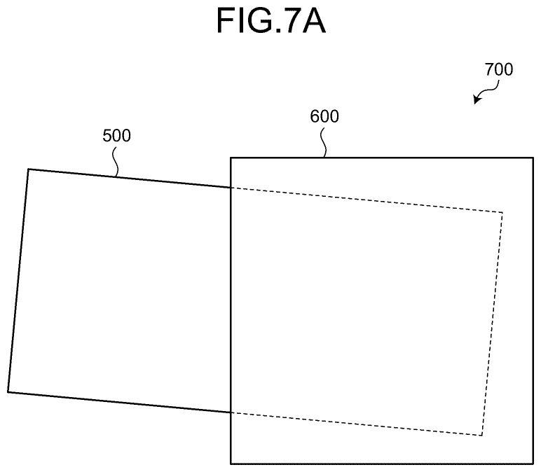

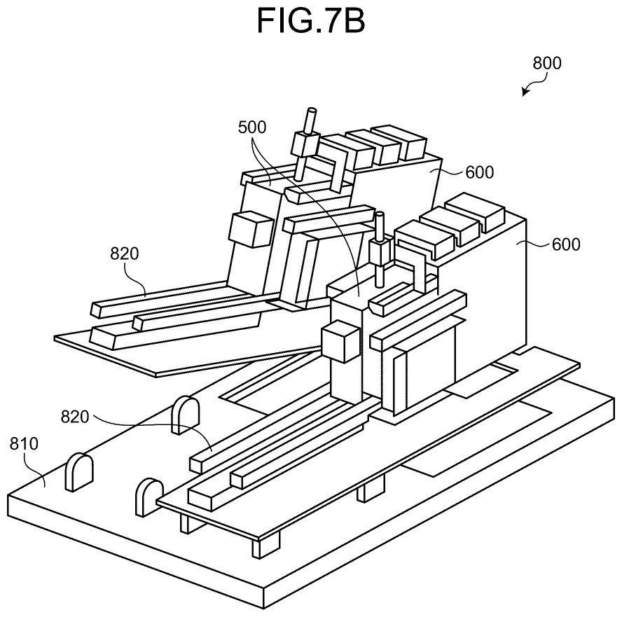

FIG. 7A is a sectional side view illustrating a configuration of a culture system 700 according to the present embodiment, and is a diagram for explaining an incubator 600 in which the cartridge 500 is housed.

As illustrated in FIG. 7A, the culture system 700 according to the present embodiment further includes the incubator 600 in which the cartridge 500 is housed. The incubator 600 is a constant-temperature bath used for cell culturing. Herein, the tilting mechanism tilts the cartridge 500.

FIG. 7A is a diagram illustrating the state in which the entire cartridge 500 is tilted. For example, the tilting mechanism tilts the culture apparatus 100 by supplying air to a balloon member (not illustrated) disposed in the bottom face of the cartridge 500. Thus, in the culture system 700 according to the present embodiment, in the state in which the culture apparatus 100 is tilted, the culture medium and the cells are collected using the collection unit.

FIG. 7B is a perspective view illustrating a configuration of a culture system 800 according to the present embodiment, and is a diagram for explaining the incubator 600 in which the cartridge 500 is housed.

As illustrated in FIG. 7B, the culture system 800 according to the present embodiment further includes a mount 810 and a support member 820. The support member 820 is disposed on the mount 810. The support member 820 is a rail-equipped member used for housing the culturing portion (i.e., the portion in which the culture apparatus 100 is disposed) of the cartridge 500 inside the incubator 600; and supports the incubator 600. The tilting mechanism tilts the cartridge 500 and the incubator 600.

FIG. 7B is a diagram illustrating the state in which the cartridge 500 and the incubator 600 are tilted across the entire culture system 800. For example, the tilting mechanism tilts the culture apparatus 100 by supplying air to the balloon member (not illustrated) disposed on the bottom face of the cartridge 500 and the incubator 600. Thus, in the culture system 800 according to the present embodiment, in the state in which the culture apparatus 100 is tilted, the culture medium and the cells are collected using the collection unit.

As explained above, in the culture system according to the present embodiment, the culture apparatus 100 is disposed that includes the supply unit (the tube 131 for cell seeding and medium replacement), the discharge unit (the detachment nozzle 141), the collection unit (the tube 131 for waste solution collection and the tube 131 for cell suspension liquid collection), and the housing 110. The support unit supplies the culture vessel 120 with the culture medium for cell culturing. The discharge unit discharges liquid droplets for detaching the cells housed in the culture vessel 120. The collection unit (in this case, the tube 131 for cell suspension liquid collection) collects the cells that have been detached inside the culture vessel 120. The housing 110 has the culture vessel 120, the supply unit, the discharge unit, and the collection unit disposed therein. In the state in which the supply unit has been moved away from the fluid level 120A of the culture medium, the housing 110 is tilted in such a way that the collection unit is positioned at a lower level than the supply unit; and the culture medium and the cells are collected using the collection unit. As a result, in the culture system according to the present embodiment, regarding the culturing of cells in the closed condition, the cell collection efficiency can be enhanced.

In the embodiment described above, the explanation is given about the case in which, when the culture apparatus 100 is at the horizontal level (i.e., in the pre-tilting state of the culture apparatus 100), the discharge outlet of the supply unit (the leading end of the tube 131 for cell seeding and medium replacement) has moved away from the fluid level 120A of the culture medium. Alternatively, in the embodiment described above, when the culture apparatus 100 is at the horizontal level (i.e., in the pre-tilting state of the culture apparatus 100), in the state in which the discharge outlet of the supply unit (the leading end of the tube 131 for cell seeding and medium replacement) is inside the culture medium and is in the tilted state, the discharge outlet of the supply unit can be treated to be away from the fluid level 120A.

According to at least one embodiment described above, regarding the culturing of cells in the closed condition, the cell collection efficiency can be enhanced.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A culture system comprising:
a supply unit that supplies a culture vessel with a culture medium for culturing of cells;
a discharge unit that discharges liquid droplets for detaching the cells placed in the culture vessel;
a collection unit that collects the cells which have been detached in the culture vessel; and
a housing in which the culture vessel, the supply unit, the discharge unit, and the collection unit are disposed, wherein
in a state in which the supply unit has been moved away from fluid level of the culture medium, the housing is tilted to position the collection unit at a lower level than the supply unit, and the culture medium and the cells are collected using the collection unit.

2. The culture system according to claim 1, wherein, in the housing, the supply unit and the collection unit are disposed in directions facing each other.

3. The culture system according to claim 2, wherein
the housing has a cylindrical shape, and
the supply unit and the collection unit are disposed opposite to each other across center of circle of top face of the cylindrical shape.

4. The culture system according to claim 1, wherein, in states before and after tilting, discharge outlet of the supply unit is disposed away from liquid level of the culture medium.

5. The culture system according to claim 1, wherein
after the culture medium and the cells are collected by the collection unit, the culture medium is added to the culture vessel by the supply unit, and
the added culture medium is also collected by the collection unit.

6. The culture system according to claim 1, wherein
the housing is tilted to position the collection unit at a lower level than the supply unit, and
tilt angle of the housing is equal to or greater than 1° and equal to or smaller than 60°.

7. The culture system according to claim 6, further comprising a tilting mechanism that, in a state in which the supply unit has been moved away from fluid level of the culture medium, tilts the housing to position the collection unit at a lower level than the supply unit.

8. The culture system according to claim 7, further comprising a culture apparatus that includes the supply unit, the discharge unit, the collection unit, and the housing, wherein
the tilting mechanism tilts the culture apparatus by supplying air to a balloon member disposed on bottom face of the culture apparatus.

9. The culture system according to claim 8, further comprising a cartridge that houses the culture apparatus and that includes
a flow path for supplying the culture medium to the supply unit,
a flow path for supplying the liquid droplets to the discharge unit,
a flow path for collecting the cells using the collection unit, and
a flow path for supplying the air to the balloon member.

10. The culture system according to claim 7, further comprising:
a culture apparatus that includes the supply unit, the discharge unit, the collection unit, and the housing; and
a cartridge that houses the culture apparatus and that includes
a flow path for supplying the culture medium to the supply unit,
a flow path for supplying the liquid droplets to the discharge unit, and
a flow path for collecting the culture medium and the cells using the collection unit, wherein
the tilting mechanism tilts the cartridge.

11. The culture system according to claim 10, wherein the tilting mechanism tilts the culture apparatus by supplying air to a balloon member disposed on bottom face of the cartridge.

12. The culture system according to claim 7, further comprising:
a culture apparatus that includes the supply unit, the discharge unit, the collection unit, and the housing;
a cartridge that houses the culture apparatus and that includes
a flow path for supplying the culture medium to the supply unit,
a flow path for supplying the liquid droplets to the discharge unit, and
a flow path for collecting the culture medium and the cells using the collection unit; and
an incubator that houses portion of the cartridge in which the culture apparatus is disposed, wherein
the tilting mechanism tilts the cartridge and the incubator.

13. The culture system according to claim 12, wherein the tilting mechanism tilts the culture apparatus by supplying air to a balloon member disposed on bottom face of the cartridge and the incubator.
